SEQUENCE LISTING (18 sequences)

SEQ ID NO: 1 - protein, 245 aa, Streptococcus suis
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 2 - protein, 10 aa, Streptococcus suis
PPGTVAQSAP

SEQ ID NO: 3 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 4 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGEHFDYDTVIEDVNGYVWVSYIGGSGKRNYVATGATKDGKRFGNAWGTFK

SEQ ID NO: 5 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGEQFDYDTAIIDVNGYAWVSYIGGSGKRNYVATGATKDGKRFGNAWGTFK

SEQ ID NO: 6 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGEQFDYDTVIIDVNGYVWVSYIGGSGKRNYVATGATKDGKRFGNAWGTFK

SEQ ID NO: 7 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGESFDYDTVIIDVNGYVWVSYIGGSGKRNYVATGATKDGKRFGNAWGTFK

SEQ ID NO: 8 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSNRETGTMTVTVDALNVRRAPNTSGEIVAVYKEGESFDYDTVIIDVNGYVWVSYIGGSGKRNYVATGATKDGKRFGNAWGTFK

SEQ ID NO: 9 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPDTSGEIVAVYKRGEHFDYDTVIIDVNGYVWVSYIGGSGKRNYVATGATKDGKRFGNAWGTFK

SEQ ID NO: 10 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPDTSGEIVAVYKRGESFDYDTAIIDVNGYAWVSYIGGSGKRNYVATGATKDGKRFGNAWGTFK

SEQ ID NO: 11 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSNRETGTMTVTVDALNVRRAPDTSGEIVAVYKEGESFDYDTEIIDVNGYVWVSYIGGSGKRNYVATGATKDGKRFGNAWGTFK

SEQ ID NO: 12 - protein, 245 aa, Artificial Sequence
  Description of Artificial Sequence Synthetic polypeptide
MTTVNEALNNVRAQVGSGVSVGNGECYALASWYEEMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 13 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIIDVNGYVWVSYIGGSGKRNYVATGATKDGKRFGNAWGTFK

SEQ ID NO: 14 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIIDVNGYVWVSYIGGSGKRNYVATGATKDGKRFGNAWGTFK

SEQ ID NO: 15 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTKIIDVNGYEWVSYIGGSGKRNYVATGATKDGKRFGNAWGTFK

SEQ ID NO: 16 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGEQFDYDTKIIDVNGYEWVSYIGGSGKRNYVATGATKDGKRFGNAWGTFK

SEQ ID NO: 17 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIEDVNGYVWGSYIGGSGKRNYVATGATKDGKRFGNAWGTFK

SEQ ID NO: 18 - DNA, 738 bp, Streptococcus suis
atgacaacagtaaatgaagcattaaataatgtaagagctcaggttgggtccggtgtgtctgttggcaacggcgaatgctacgctttggctagttggtacgagcgcatgattagtccggatgcaactgtcggacttggcgctggtgtgggctgggtcagcggtgcaatcggcgatacaatctctgccaaaaacatcggctcatcatacaactggcaagctaacggctggacagtttccacatctggtccatttaaagcaggtcagattgtgacgcttggggcaacaccaggaaacccttacggacatgtggtaatcgtcgaagcagtggacggcgatagattgactattttggagcaaaactacggcgggaaacgttatcccgtccgtaattattacagcgctgcaagctatcgtcaacaggtcgtgcattacatcacaccgcctggcacggtcgcacagtcagcacccaaccttgcaggctctcgttcctatcgcgagacgggcactatgactgtcacggtcgatgctctcaatgttcgcagggggccaaatacttcaggcgagattgtagcagtatacaagcgtggtgaatcatttgactatgatactgtcatcatcgatgtcaatggctatgtctgggtgtcttacataggcggcagcggcaaacgtaactacgttgcgacgggcgctaccaaagacggtaagcgtttcggcaatgcttggggtacatttaaataa

(19) United States
(12) Patent Application Publication
(10) Pub. No.: US 2022/0193186 A1
(43) Pub. Date: Jun. 23, 2022

(54) METHOD OF TREATING AND PREVENTING BONE AND JOINT INFECTIONS

(71) Applicant: CONTRAFECT CORPORATION, Yonkers, NY (US)

(72) Inventor: Raymond SCHUCH, Mountain Lakes, NJ (US)

(21) Appl. No.: 17/602,064

(22) PCT Filed: Apr. 10, 2020
(86) PCT No.: PCT/US2020/027752

§ 371 Date: Oct. 7, 2021

Related U.S. Application Data
(60) Provisional application No. 62/832,754, filed on Apr. 11, 2019.
(60) Provisional application No. 62/849,672, filed on May 17, 2019.
(60) Provisional application No. 62/938,812, filed on Nov. 21, 2019.
(60) Provisional application No. 62/964,755, filed on Jan. 23, 2020.

(51) Int. Cl. A61K 38/12; A61K 38/47; A61K 45/06; A61P 19/02; A61P 19/08

(52) U.S. Cl. CPC A61K 38/12; A61K 38/47; A61P 19/08; A61P 19/02; A61K 45/06

(57) ABSTRACT

The present disclosure is directed to a method of treating or preventing a bone or joint infection, which method comprises: administering a therapeutically effective amount of a PlySs2 lysin comprising the amino acid sequence of SEQ ID NO: 1 or a variant thereof having at least 80% identity to SEQ ID NO: 1, wherein the variant comprises bacteriocidal and/or bacteriostatic activity against the Gram-positive bacteria, to a subject in need thereof, optionally co-administered with one or more antibiotics, wherein the bone or joint infection comprises a Gram-positive bacteria, such as Staphylococcus epidermidis or Staphylococcus aureus. Methods for preventing or disrupting a Gram-positive bacterial biofilm formed in a synovial fluid, such as a biofilm formed by Staphylococcus epidermidis, are also disclosed.

### CROSS-REFERENCE TO RELATED APPLICATIONS

This application claims the benefit of, and relies on the filing date of, U.S. provisional patent application No. 62/832,754, filed 11 Apr. 2019, U.S. provisional patent application No. 62/849,672, filed 17 May 2019, U.S. provisional patent application No. 62/938,812, filed 21 Nov. 2019 and U.S. provisional patent application No. 62/964,755 filed 23 Jan. 2020, the entire disclosures of each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on 10 Apr. 2020, is named 0341_0020-00-304_ST25.txt and is 37,401 bytes in size.

### FIELD OF THE DISCLOSURE

The present disclosure relates generally to the treatment and prevention of bone and joint infections, particularly osteomyelitis and prosthetic joint infections due to Gram-positive bacteria, such as Staphylococcus aureus and Staphylococcus epidermidis, using lysin(s) and optionally one or more antibiotics.

### BACKGROUND

Microorganisms can be categorized into two different life forms, namely the planktonic and the biofilm form. Planktonic microorganisms are free-floating, have an active metabolism and replicate rapidly. In contrast, biofilm microorganisms exist as multicellular, complex three dimensional structures. They are in a stationary phase of growth and are metabolically less active.

Typically, biofilms form in “stages,” including attachment of microbial cells to a surface, such as a host cell surface, followed by cellular aggregation, maturation, and subsequent detachment. During initial attachment, host proteins such as fibrinogen, fibronectin, and vitronectin are absorbed onto the surface, resulting in the formation of a conditioning film. The absorbed host proteins enhance, e.g., bacterial colonization, through interactions between bacterial proteins and host proteins.

After initial cell attachment onto a surface, multilayer cellular proliferation occurs, as well as cell-to-cell adhesion, culminating in the formation of microcolonies of one or several species. This stage is followed by maturation wherein the adhered cells grow and interact amongst themselves. At this stage, bacterial cells, for example, start secretion of exopolysaccharides that enclose the cells and stabilize the biofilm network. Upon maturation, large biofilms may release planktonic forms from their surfaces, which then disperse to cause further local invasion or seeding of distant sites, thus initiating an entirely new cycle.

Many difficult-to-treat infections are biofilm infections, such as many bone infections and those associated with implant material, e.g., prosthetic joints. In these infections, microorganisms typically adhere either onto dead bone or implants and form biofilms, which withstand not only host mechanisms but also most antimicrobial agents. Accordingly, antibiotics often exhibit poor activity against bone and joint infections, thus requiring prolonged courses of antibiotic therapy, usually in combination with surgery, before such treatment is effective.

In view of the above, novel strategies are needed to treat bone and joint infections due to biofilm-forming bacteria. These strategies should include drugs and/or biologics that are capable of both eradicating biofilms as well as killing biofilm-forming bacteria.

### SUMMARY

In one aspect, the present disclosure is directed to a method of treating or preventing a bone or joint infection, such as osteomyelitis, e.g. acute osteomyelitis, which method comprises: administering a therapeutically effective amount of a PlySs2 lysin or a variant thereof as described herein to a subject in need thereof, wherein the bone or joint infection comprises a Gram-positive bacteria.

The present disclosure is also directed to a method for prevention or disruption of a biofilm formed in a synovial fluid of a subject comprising: administering a therapeutically effective amount of a PlySs2 lysin or a variant thereof as described herein to a subject in need thereof, wherein the biofilm is formed by a Gram-positive bacteria.

### BRIEF DESCRIPTION OF THE DRAWINGS

FIG. 1 depicts the amino acid sequence of a lysin (SEQ ID NO: 1) and a polynucleotide (SEQ ID NO: 18) encoding the lysin as described in the detailed description. SEQ ID NO: 1 represents a 245 amino acid polypeptide, including the initial methionine residue which is removed during post-translational processing, leaving a 244-amino acid polypeptide.

FIG. 2 depicts the impact of lysin treatment on ethidium bromide stained biofilm structures formed by Staphylococcus epidermidis in human synovial fluid as described in the Examples.

FIG. 3 depicts fluorescent images of biofilms formed in human synovial fluid before and after treatment with the PlySs2lysin (also referred to herein as CF-301 and exebacase) as described in the Examples.

FIG. 4 depicts a Scanning Electron Micrograph showing biofilm disruption in human synovial fluid after treatment with a PlySs2lysin as described in the Examples.

FIG. 5 depicts quantitative bacterial cultures of rat tibias in log₁₀ cfu/gram of bone after treatment with the exebacase lysin, either alone, or in combination with daptomycin as described in the Examples.

FIGS. 6A-6C depict the condition of patients with infected prosthetic knees who were selected for treatment using the present methods as described in Example 5. FIG. 6A is an X-ray showing the patients' prosthetic knees. FIG. 6B depicts the clinical signs of septic arthritis observed in two of the selected patients. FIG. 6C depicts the favorable outcome of the septic arthritic patients after treatment.

### DETAILED DESCRIPTION

### Definitions

As used herein, the following terms and cognates thereof shall have the following meanings unless the context clearly indicates otherwise:

“Carrier” refers to a solvent, additive, excipient, dispersion medium, solubilizing agent, coating, preservative, isotonic and absorption delaying agent, surfactant, propellant, diluent, vehicle and the like with which an active compound is administered. Such carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like.

“Pharmaceutically acceptable carrier” refers to any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in “Remington's Pharmaceutical Sciences” by E.W. Martin, 18th Edition. The pharmaceutically acceptable carrier may be a carrier that does not exist in nature.

“Bactericidal” or “bactericidal activity” refers to the property of causing the death of bacteria or capable of killing bacteria to an extent of at least a 3−log 10 (99.9%) or a better reduction among an initial population of bacteria over an 18-24 hour period.

“Bacteriostatic” or “bacteriostatic activity” refers to the property of inhibiting bacterial growth, including inhibiting growing bacterial cells, thus causing a 2−log 10 (99%) or better and up to just under a 3−log reduction among an initial population of bacteria over an 18-24 hour period.

“Antibacterial” refers to both bacteriostatic and bactericidal agents.

“Antibiotic” refers to a compound having properties that have a negative effect on bacteria, such as lethality or reduction of growth. An antibiotic can have a negative effect on Gram-positive bacteria, Gram-negative bacteria, or both. By way of example, an antibiotic can affect cell wall peptidoglycan biosynthesis, cell membrane integrity or DNA or protein synthesis in bacteria.

“Drug resistant” refers generally to a bacterium that is resistant to the antibacterial activity of a drug. When used in certain ways, drug resistance may specifically refer to antibiotic resistance. In some cases, a bacterium that is generally susceptible to a particular antibiotic can develop resistance to the antibiotic, thereby becoming a drug resistant microbe or strain. A “multi-drug resistant” (“MDR”) pathogen is one that has developed resistance to at least two classes of antimicrobial drugs, each used as monotherapy. For example, certain strains of S. aureus have been found to be resistant to several antibiotics including methicillin and/or vancomycin (Antibiotic Resistant Threats in the United States, 2013, U.S. Department of Health and Services, Centers for Disease Control and Prevention). One skilled in the art can readily determine if a bacterium is drug resistant using routine laboratory techniques that determine the susceptibility or resistance of a bacterium to a drug or antibiotic.

“Effective amount” refers to an amount which, when applied or administered in an appropriate frequency or dosing regimen, is sufficient to prevent, reduce, inhibit or eliminate bacterial growth or bacterial burden or prevent, reduce or ameliorate the onset, severity, duration or progression of the disorder being treated (here Gram-positive bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” refers to the administration of two agents, such as a lysin, and an antibiotic or any other antibacterial agent in a sequential manner, as well as administration of these agents in a substantially simultaneous manner, such as in a single mixture/composition or in doses given separately, but nonetheless administered substantially simultaneously to the subject, for example at different times in the same day or 24-hour period. Such co-administration of two agents, such as a lysin with one or more additional antibacterial agents, can be provided as a continuous treatment lasting up to days, weeks, or months. Additionally, depending on the use, the co-administration need not be continuous or coextensive. For example, if the use were as a systemic antibacterial agent to treat, e.g., a joint or bone infection, the lysin, could be administered only initially within 24 hours of an additional antibiotic use and then the additional antibiotic use may continue without further administration of the lysin.

“Subject” refers to a mammal, a plant, a lower animal, a single cell organism or a cell culture. For example, the term “subject” is intended to include organisms, e.g., prokaryotes and eukaryotes, which are susceptible to or afflicted with bacterial infections, for example Gram-positive bacterial infections. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to infection by Gram-positive bacteria, whether such infection be systemic, topical or otherwise concentrated or confined to a particular organ or tissue.

“Polypeptide” refers to a polymer made from amino acid residues and generally having at least about 30 amino acid residues. The term “polypeptide” is used herein interchangeably with the term “protein” and “peptide.” The term includes not only polypeptides in isolated form, but also active fragments and derivatives thereof. The term “polypeptide” also encompasses fusion proteins or fusion polypeptides comprising a lysin polypeptide, and maintaining, for example, a lysin function. Depending on context, a polypeptide or protein or peptide can be a naturally occurring polypeptide or a recombinant, engineered or synthetically produced polypeptide. A particular lysin polypeptide, for example, can be, e.g., derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, maintaining e.g., lysin activity against the same or at least one common target bacterium.

“Fusion polypeptide” refers to an expression product resulting from the fusion of two or more nucleic acid segments, resulting in a fused expression product typically having two or more domains or segments, which typically have different properties or functionality. In a more particular sense, the term “fusion polypeptide” also refers to a polypeptide or peptide comprising two or more heterologous polypeptides or peptides covalently linked, either directly or via an amino acid or peptide linker. The polypeptides forming the fusion polypeptide are typically linked C-terminus to N-terminus, although they can also be linked C-terminus to C-terminus, N-terminus to N-terminus, or N-terminus to C-terminus. The term “fusion polypeptide” can be used interchangeably with the term “fusion protein. Thus, the open-ended expression “a polypeptide comprising” a certain structure includes larger molecules than the recited structure such as fusion polypeptides.

“Heterologous” refers to nucleotide or polypeptide sequences that are not naturally contiguous. For example, in the context of the present disclosure, the term “heterologous” can be used to describe a combination or fusion of two or more polypeptides wherein the fusion polypeptide is not normally found in nature, such as for example a lysin polypeptide and a cationic and/or a polycationic peptide, an amphipathic peptide, a sushi peptide (Ding et al. Cell Mol Life Sci., 65(7-8):1202-19 (2008)), a defensin peptide (Ganz, T. Nature Reviews Immunology 3, 710-720 (2003)), a hydrophobic peptide, and/or an antimicrobial peptide which may have enhanced lytic activity. Included in this definition are two or more lysin polypeptides or active fragments thereof. These can be used to make a fusion polypeptide with lytic activity.

“Active fragment” refers to a portion of a polypeptide that retains one or more functions or biological activities of the isolated polypeptide from which the fragment was taken, for example bactericidal activity against one or more Gram-positive bacteria, such as S. aureus or S. epidermidis.

“Synergistic” or “Superadditive” refers to a beneficial effect brought about by two substances in combination that exceeds the sum of the effects of the two agents working independently. In certain embodiments the synergistic or superadditive effect significantly, i.e., statistically significantly, exceeds the sum of the effects of the two agents working independently. One or both active ingredients may be employed at a sub-threshold level, i.e., a level at which if the active substance is employed individually produces no or a very limited effect. The effect can be measured by assays such as the checkerboard assay, described here.

“Treatment” refers to any process, action, application, therapy, or the like, wherein a subject, including a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis or combinations thereof. “Treatment” may further encompass reducing the population, growth rate or virulence of the bacteria in the subject and thereby controlling or reducing a bacterial infection in a subject or bacterial contamination of an organ, tissue or environment. Thus, “treatment” that reduces incidence may, for example, be effective to inhibit growth of at least one Gram-positive bacterium in a particular milieu, whether it be a subject or an environment. On the other hand “treatment” of an already established infection refers to reducing the population, killing, inhibiting the growth, and/or eradicating, the Gram-positive bacteria responsible for an infection or contamination.

“Preventing” refers to the prevention of the incidence, recurrence, spread, onset or establishment of a disorder such as a bacterial infection. It is not intended that the present disclosure be limited to complete prevention or to prevention of establishment of an infection. In some embodiments, the onset is delayed, or the severity of a subsequently contracted disease or the chance of contracting the disease is reduced, and such constitutes examples of prevention.

“Contracted diseases” refers to diseases manifesting with clinical or subclinical symptoms, such as the detection of fever, sepsis or bacteremia, as well as diseases that may be detected by growth of a bacterial pathogen (e.g., in culture) when symptoms associated with such pathology are not yet manifest.

“Derivative,” in the context of a peptide or polypeptide or active fragment thereof, is intended to encompass, for example, a polypeptide modified to contain one or more-chemical moieties other than an amino acid that do not substantially adversely impact or destroy the polypeptide's activity, such as lysin activity. The chemical moiety can be linked covalently to the peptide, e.g., via an amino terminal amino acid residue, a carboxy terminal amino acid residue, or at an internal amino acid residue. Such modifications may be natural or non-natural. In certain embodiments, a non-natural modification may include the addition of a protective or capping group on a reactive moiety, addition of a detectable label, such as an antibody and/or fluorescent label, addition or modification of glycosylation, or addition of a bulking group such as PEG (pegylation) and other changes known to those skilled in the art. In certain embodiments, the non-natural modification may be a capping modification, such as N-terminal acetylations and C-terminal amidations. Exemplary protective groups that may be added to lysin polypeptides include, but are not limited to t-Boc and Fmoc. Commonly used fluorescent label proteins such as, but not limited to, green fluorescent protein (GFP), red fluorescent protein (RFP), cyan fluorescent protein (CFP), yellow fluorescent protein (YFP) and mCherry, are compact proteins that can be bound covalently or noncovalently to a polypeptide or fused to a polypeptide without interfering with normal functions of cellular proteins. In certain embodiments, a polynucleotide encoding a fluorescent protein is inserted upstream or downstream of the polynucleotide sequence. This will produce a fusion protein (e.g., Lysin Polypeptide::GFP) that does not interfere with cellular function or function of a polypeptide to which it is attached. Polyethylene glycol (PEG) conjugation to proteins has been used as a method for extending the circulating half-life of many pharmaceutical proteins. Thus, in the context of polypeptide derivatives, such as lysin polypeptide derivatives, the term “derivative” encompasses polypeptides, such as lysin polypeptides, chemically modified by covalent attachment of one or more PEG molecules. It is anticipated that lysin polypeptides, such as pegylated lysins, will exhibit prolonged circulation half-life compared to unpegylated polypeptides, while retaining biological and therapeutic activity.

“Percent amino acid sequence identity” refers to the percentage of amino acid residues in a candidate sequence that are identical with the amino acid residues in the reference polypeptide sequence, such as a lysin polypeptide sequence, after aligning the sequences and introducing gaps, if necessary, to achieve the maximum percent sequence identity, and not considering any conservative substitutions as a part of the sequence identity. Alignment for purposes of determining percent amino acid sequence identity can be achieved in various ways that are within the skill in the art, for example, using publicly available software such as BLAST or software available commercially for example from DNASTAR. Two or more polypeptide sequences can be anywhere from 0-100% identical, or any integer value there between. In the context of the present disclosure, two polypeptides are “substantially identical” when at least 80% of the amino acid residues (typically at least about 85%, at least about 90%, and typically at least about 95%, at least about 98%, or at least 99%) are identical. The term “percent (%) amino acid sequence identity” as described herein applies to peptides as well. Thus, the term “Substantially identical” will encompass mutated, truncated, fused, or otherwise sequence-modified variants of isolated polypeptides and peptides, such as those described herein, and active fragments thereof, as well as polypeptides with substantial sequence identity (e.g., at least 80%, at least 85%, at least 90%, at least 95% identity, at least 98% identity, or at least 99% identity as measured for example by one or more methods referenced above) as compared to the reference (wild type or other intact) polypeptide. Two amino acid sequences are “substantially homologous” when at least about 80% of the amino acid residues (typically at least about 85%, at least about 90%, at least about 95%, at least about 98% identity, or at least about 99% identity) are identical, or represent conservative substitutions. The sequences of polypeptides of the present disclosure, are substantially homologous when one or more, or several, or up to 10%, or up to 15%, or up to 20% of the amino acids of the polypeptide, such as the lysin polypeptides described herein, are substituted with a similar or conservative amino acid substitution, and wherein the resulting polypeptide, such as the lysins described herein, have at least one activity, antibacterial effects, and/or bacterial specificities of the reference polypeptide, such as the lysins described herein.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Biofilm” refers to bacteria that attach to surfaces and aggregate in a hydrated polymeric matrix that may be comprised of bacterial- and/or host-derived components. A biofilm is an aggregate of microorganisms in which cells adhere to each other on a biotic or abiotic surface. These adherent cells are frequently embedded within a matrix comprised of, but not limited to, extracellular polymeric substance (EPS). Biofilm EPS, which is also referred to as slime (although not everything described as slime is a biofilm) or plaque, is a polymeric conglomeration generally composed of extracellular DNA, proteins, and polysaccharides.

“Suitable” in the context of an antibiotic being suitable for use against certain bacteria refers to an antibiotic that was found to be effective against those bacteria even if resistance subsequently developed.

Bone and Joint Infections

The present inventor has surprisingly recognized that certain biologics, i.e., lysins, may be used to kill biofilm-forming bacteria that may cause bone and joint infections, such as Staphylococcus epidermidis and Staphylococcus aureus including methicillin-resistant Staphylococcus aureus (MRSA) and multiple drug resistant (MDR) Staphylococcus epidermidis. Lysins are also surprisingly able to disrupt mature biofilms formed by, e.g. Staphylococcus epidermidis or Staphylococcus aureus, in synovial fluid or bone. These anti-microbial agents are bacteriophage-encoded hydrolytic enzymes that liberate progeny phage from infected bacteria by degrading peptidoglycan from inside the cell, causing lysis of the host bacterium. Lysins act against pathogenic bacteria by attacking peptidoglycan from outside the bacterial cell. Typically, lysins are highly specific for bacterial species and rarely lyse non-target organisms, including commensal gut bacteria, which may be beneficial in maintaining gastrointestinal homeostasis.

In one aspect, the present disclosure is directed to a method of treating a bone or joint infection, which method comprises: administering a therapeutically effective amount of a PlySs2 lysin as described herein to a subject in need thereof, wherein the bone or joint infection comprises a Gram-positive bacteria.

In some embodiments, the bone infection is osteomyelitis, i.e., an inflammatory reaction of bone to an infecting organism. In some embodiments, the bone infection, such as osteomyelitis is due to Gram-positive bacteria, such as Staphylococcus aureus or MRSA. In some embodiments, the gram positive bacteria has the ability to form biofilms and to enter into and survive within osteoblasts, thus allowing the Gram-positive bacteria to evade the immune system and many traditional antibiotics.

In some embodiments, the osteomyelitis is acute osteomyelitis. In other embodiments, the bone infection is chronic osteomyelitis. Osteomyelitis is considered chronic when the delay between infection and efficacious treatment exceeds 4-6 weeks.

In some embodiments, the osteomyelitis comprises an infection of a long bone, such as the femur, tibia, humerus, and radius. In other embodiments, the osteomyelitis comprises an infection of the vertebral column, in particular the lumbar spine, the sacrum, and the pelvis. Typically, children develop osteomyelitis in long bone and adults develop osteomyelitis in the vertebral column.

In some embodiments, the osteomyelitis is exogenous osteomyelitis. In these embodiments, the exogenous osteomyelitis may occur when bone extends out from the skin, allowing a potentially infectious organism to enter from an abscess or burn, a puncture wound, or other trauma such as an open fracture. In other embodiments, the exogenous osteomyelitis is implant-associated osteomyelitis. Typically, the implant is a mechanical device, such as a metal plate, pin, rod, wire or screw, which is used, e.g. to stabilize and join the ends of fractured bones. In some embodiments, implant-associated osteomyelitis becomes chronic when only antibiotics are used to treat the infection.

In some embodiments, the osteomyelitis is haematogenous osteomyelitis. Haematogenous osteomyelitis may be acquired from the spread of organisms from preexisting infections e.g., impetigo, furunculosis (persistent boils), infected lesions of varicella (chickenpox), and sinus, ear, dental, soft tissue, respiratory, and genitourinary infections. In some embodiments, a genitourinary infection can lead to osteomyelitis of the sacrum or iliac.

In some embodiments, chronic osteomyelitis occurs in patients who suffered from acute osteomyelitis in the pre-antibiotic era or in their childhood. Such infections can recur after a symptom-free interval of several decades due to, e.g., the asymptomatic persistence of a biofilm adhering on dead bone.

In other embodiments, the lysins of the present methods are used to treat a joint infection. Infected joints may include infected hip, knee, ankle, shoulder, elbow or wrist joints. Typically, the infected joint is a knee joint or a hip joint.

In some embodiments, the infected joint is a native joint. Infection of a native joint (also referred to herein as septic arthritis of a native joint) may occur when a penetrating injury, such as a puncture wound, occurs near or above a joint, allowing bacteria to directly enter the joint. In other embodiments, the joint infection occurs when bacteria from a distant infection spreads through the bloodstream to the native joint.

In other embodiments, the infected joint is a prosthetic joint, including, for example, septic arthritis of a prosthetic joint). The prosthetic joints may include hip, knee, shoulder, elbow, and ankle prostheses. Typically, the prosthetic joint is a prosthetic hip or knee.

In some embodiments, the prosthetic joint infection of the present disclosure occurs within 1 year of surgery. Such an infection can be initiated through the introduction of microorganisms at the time of surgery. This can occur through either direct contact or aerosolized contamination of the prosthesis or periprosthetic tissue. Once in contact with the surface of the implant, microorganisms may colonize the surface.

In other embodiments, the prosthetic joint infections occur due to the spread of an infection from an adjacent site. For example, in the early postoperative time period, a superficial surgical site infection can progress to involve the prosthesis. In other embodiments, the prosthetic joint infection occurs due to the spread of organisms from a remote site of infection via the bloodstream.

In some embodiments, the prosthetic joint infection is recurring. For example, in some embodiments, the joint infection is a relapsing multiple drug resistant infection, such as a relapsing multiple drug resistant S. epidermidis prosthetic knee infection (PKI).

Typically, a prosthetic joint infection is indicated when a pathogen is isolated by culture from at least two separate tissue or fluid samples obtained from the affected prosthetic joint or when four of the following six criteria exist: elevated serum erythrocyte sedimentation rate (ESR) and serum C-reactive protein (CRP) concentration, elevated synovial leukocyte count, elevated synovial neutrophil percentage (PMN %), presence of purulence in the affected joint, isolation of a microorganism in one culture of periprosthetic tissue or fluid, or greater than five neutrophils per high-power field in five high-power fields observed from histologic analysis of periprosthetic tissue at ×400 magnification.

Typically, the fluid obtained from a prosthetic joint to assess for pathogens is synovial fluid. As used herein, “synovial fluid” is a viscous fluid found in the cavities of synovial joints. The principal role of synovial fluid is to reduce friction between the articular cartilage of synovial joints during movement.

In some embodiments, a synovial fluid sample can be obtained by aspiration. The aspirant may be assessed for total nucleated cell counts and neutrophil percentages as an indicator of prosthetic joint infection. Typically, the amount of total nucleated cells per microliter and/or the percentage of neutrophils is greater in a synovial fluid obtained from a subject suffering from prosthetic joint infection in comparison to that of a subject who is not suffering from a prosthetic joint infection. For example, in some embodiments, a threshold of 1,100 total nucleated cells per microliter and/or a threshold of 64% neutrophils in a synovial fluid from a subject with a prosthetic joint indicates a prosthetic joint infection, such as a prosthetic knee joint infection.

In some embodiments, instead of or in addition to determining an amount of neutrophils, a level of leukocyte esterase, an enzyme present in neutrophils, may be assessed using, e.g., colorimetric strips that are widely available for determining pyruia for the diagnosis of urinary tract infection as described in Parvizi et al., “Diagnosis of periprosthetic joint infection: the utility of a simple yet unappreciated enzyme.”, J. Bone Joint Surg. Am., 2011, 93:2242-2248, which is herein incorporated by reference in its entirety.

More typically, however, a synovial fluid sample is cultured to determine whether or not a diagnosis of prosthetic joint infection is indicated and to identify the infecting pathogen(s). This information can also inform the choice of antibiotics if used during treatment. In these embodiments, aspirated synovial fluid can be either inoculated into blood culture bottles at the time of collection or transported to a microbiology laboratory and inoculated onto solid and/or liquid media. See, e.g., Fehring et al., “Aspiration as a guide to sepsis in revision total hip arthroplasty,” 1996, J. Arthroplasty, 11:543-547, which is herein incorporated by reference in its entirety.

Causative Microorganisms

In some embodiments, the present bone and/or joint infections are caused by Gram-positive bacteria, such as a Streptococcus species including Streptococcus gallolyticus and Streptococcus pneumonia. More typically, however, the bone and/or joint infection is caused by a Staphylococcus species e.g. S. aureus or S. epidermidis. In other embodiments, the Staphylococcus species is a coagulase-negative Staphylococcus species such as Staphylococcus epidermidis, Staphylococcus simulans, Staphylococcus caprae, Staphylococcus lugdunensis or a combination thereof. Typically, Staphylococcus epidermidis is the coagulase-negative Staphylococcus species identified in bone and/or joint infections.

In some embodiments, the present bone and/or joint infections are caused by Gram-positive bacterial species from the Enterococcus genus.

In some embodiments, the present bone and/or joint infections are caused by a polymicrobial infection. For example, a combination of Enterococcus species and Staphylococcus species may be identified as causative agents of a bone and/or joint infection. Examples of causative microorganisms, typically associated with specific infected structures are shown below in Table 1.

TABLE 1
Gram-positive bacterial causative agents of bone and
joint infections

| | STRUCTURE | |
|---|---|---|
| HISTORY | INFECTED | CAUSATIVE AGENT(S) |
| Acute | Bone | |
| | Long Bones | Staphylococcus aureus |
| | Spine | Staphylococcus aureus |
| | Any bone following | Staphylococcus aureus |
| | insertion of implant | Coagulase-negative staphylococcus |
| | Any bone following | Staphylococcus aureus |
| | open fracture | Coagulase-negative staphylococcus |
| | Joint | |
| | Native | Staphylococcus aureus |
| | | Coagulase-negative staphylococcus |
| | Prosthetic | Staphylococcus aureus |
| | | Coagulase-negative staphylococcus |
| | | Enterococci |
| Chronic | Bone | |
| | Spine | Staphylococcus aureus |
| | | Coagulase-negative staphylococcus |
| | Long Bone | Staphylococcus aureus |
| | | Coagulase-negative staphylococcus |
| | Pelvis | Staphylococcus aureus |
| | | Coagulase-negative staphylococcus |
| | | Enterococci |
| | Joint | |
| | Native | Staphylococcus aureus |
| | Prosthetic | Staphylococcus aureus |
| | | Coagulase-negative staphylococcus |
| | | Enterococci |

Lysins

The present methods for treating and/or preventing bone and joint infections and/or inhibiting or disrupting biofilm formation in a subject, comprise administering a lysin or active fragment thereof or a variant or derivative thereof as described herein to a subject in need thereof, optionally in combination with one or more antibiotics as also herein described. In some embodiments, the present lysins or active fragments thereof or variants or derivatives thereof exhibit bacteriocidal and/or bacteriostatic activity against Gram-positive bacteria. In some embodiments, the present lysins or active fragments thereof or variants or derivatives thereof also exhibit a low propensity for resistance, suppress antibiotic resistance and/or exhibit synergy with conventional antibiotics. In other embodiments, the present lysins or active fragments thereof or variants or derivatives thereof inhibit bacterial agglutination, biofilm formation and/or reduce or eradicate biofilm, including biofilm in a subject with a bone or joint infection.

The bacteriocidal activity of the present lysins or active fragments thereof or variants or derivatives thereof may be determined using any method known in the art. For example, the present lysins or active fragments thereof or variants or derivatives thereof may be assessed in vitro using time kill assays as described, for example, in Mueller, et al., 2004, Antimicrob Agents Chemotherapy, 48:369-377, which is herein incorporated by reference in its entirety.

The bacteriostatic activity of the present lysins or active fragments thereof or variants or derivatives thereof may also be assessed using any art-known method. For example, growth curves may be performed in e.g., cation adjusted Mueller Hinton II Broth supplemented in human serum (caMHB/50% HuS) to a final concentration of 50% or in 100% serum or in a non-standard medium (caMHB supplemented to 25% with horse serum and 0.5 mM with DTT (caMHB-HSD)). The Gram-positive bacteria may be suspended with lysin and culture turbidity can be measured at an optical density at 600 nm using, e.g. a SPECTRAMAX® M3 Multi-Mode Microplate reader (Molecular Devices) with e.g., readings every 1 minute for 11 hours at 24° C. with agitation. Doubling times can be calculated in the logarithmic-phase of cultures grown in flasks with aeration according to the method described in Saito et al, 2014, Antimicrob Agents Chemother 58:5024-5025, which is herein incorporated by reference in its entirety and compared to the doubling times of cultures in the absence of the present lysins or active fragments thereof or variants or derivatives thereof.

In some embodiments, the present lysins or active fragments thereof or variants or derivatives thereof exhibit lysin activity in the presence of synovial fluid, such as human synovial fluid. Suitable methods for assessing the activity of a lysin in synovial fluid are known in the art and described in the examples. Briefly, a MIC value (i.e., the minimum concentration of peptide sufficient to suppress at least 80% of the bacterial growth compared to control) may be determined for a lysin in a synovial fluid and its MIC value compared to, e.g., a parent lysin or the absence of lysin.

More particularly MIC values for a lysin may be determined against e.g., S. epidermidis or S. aureus in e.g., Mueller-Hinton broth (MHB) supplemented with physiological salt concentrations and synovial fluid, such as human synovial fluid. Minimum Inhibitory Concentrations (MICs) of a lysin against e.g., S. epidermidis may be determined using broth microdilution (BMD) following Clinical and Laboratory Standards Institute (CLSI) methodology (M07-A11, 2018, which is herein incorporated by reference in its entirety) in a non-standard medium (caMHB supplemented to 50% with human synovial fluid (caMHB-HSF)). See Examples.

In some embodiments, the present isolated polypeptides comprising lysins, variant lysins, active fragments thereof or derivatives reduce the minimum inhibitory concentration (MIC) of an antibiotic needed to inhibit bacteria in the presence of e.g., human serum or synovial fluid. Any known method to assess this MIC may be used. In some embodiments, a checkerboard assay is used to determine the effect of a lysin on antibiotic concentration. The checkerboard assay is based on a modification of the CLSI method for MIC determination by broth microdilution (See CLSI. 2015. Methods for Dilution Antimicrobial Susceptibility Tests for Bacteria That Grow Aerobically; Approved Standard-10th Edition. Clinical and Laboratory Standards Institute, Wayne, Pa., which is herein incorporated by reference in its entirety and Ceri et al. 1999. J. Clin. Microbiol. 37: 1771-1776, which is also herein incorporated by reference in its entirety).

Checkerboards are constructed by first preparing columns of e.g., a 96-well polypropylene microtiter plate, wherein each well has the same amount of antibiotic diluted 2-fold along the horizontal axis. In a separate plate, comparable rows are prepared in which each well has the same amount of lysin diluted e.g., 2-fold along the vertical axis. The lysin and antibiotic dilutions are then combined, so that each column has a constant amount of antibiotic and doubling dilutions of lysin, while each row has a constant amount of lysin and doubling dilutions of antibiotic. Each well thus has a unique combination of lysin and antibiotic. Bacteria are added to the drug combinations at concentrations of 1×10⁵ CFU/ml in caMHB-HSF, for example. The MIC of each drug, alone and in combination, is then recorded after e.g., 16 hours at 37° C. in ambient air. Summation fractional inhibitory concentrations (ΣFICs) are calculated for each drug and the minimum ΣFIC value (ΣFICmin) is used to determine the effect of the lysin/antibiotic combination.

Inhibition of bacterial agglutination may be assessed using any method known in the art. For example, the method described in Walker et al. may be used, i.e., Walker et al., 2013, PLoS Pathog, 9:e1003819, which is herein incorporated by reference in its entirety.

Methods for assessing the ability of the lysins or active fragments thereof or variants or derivatives thereof to inhibit or reduce biofilm formation in vitro are well known in the art and include a variation of the broth microdilution minimum Inhibitory Concentration (MIC) method with modifications (See Ceri et al. 1999. J. Clin Microbial. 37:1771-1776, which is herein incorporated by reference in its entirety and Schuch et al., 2017, Antimicrob. Agents Chemother. 61, pages 1-18, which is herein incorporated by reference in its entirety.) In this method for assessing the Minimal Biofilm Eradicating Concentration (MBEC), fresh colonies of e.g., an S. aureus strain or an S. epidermidis strain, are suspended in medium, e.g., phosphate buffer solution (PBS) diluted e.g., 1:100 in TSBg (tryptic soy broth supplemented with 0.2% glucose), added as e.g., 0.15 ml aliquots, to a Calgary Biofilm Device (96-well plate with a lid bearing 96 polycarbonate pegs; Innovotech Inc.) and incubated e.g., 24 hours at 37° C. Biofilms are then washed and treated with e.g., a 2-fold dilution series of the lysin in e.g., TSBg at e.g., 37° C. for 24 hours. After treatment, wells are washed, air-dried at e.g., 37° C. and stained with e.g., 0.05% crystal violet for 10 minutes. After staining, the biofilms are destained in e.g., 33% acetic acid and the OD600 of e.g., extracted crystal violet is determined. The MBEC of each sample is the minimum lysin concentration required to remove >95% of the biofilm biomass assessed by crystal violet quantitation.

In some embodiments, the present lysins, variant lysins and fragments thereof are assessed against a Gram-positive bacterial lysate obtained from a subject with a bone and/or joint infection as described herein. Methods for obtaining such isolates are well known in the art and described, for example, in Schmidt-Malan et al., Diag. Microbiol. Infect. Dis. 85:77-79, which is herein incorporated by reference in its entirety.

Suitable lysins for use with the present method include the PlySs2 lysins as described in WO 2013/170015 and WO 2013/170022, each of which is herein incorporated by reference in its entirety. As used herein, the terms “PlySs2 lysin”, “PlySs2 lysins”, “PlySs2” “Exebacase” and “CF-301” are used interchangeably and encompass the PlySs2 lysin set forth herein as SEQ ID NO: 1 (with or without initial methionine residue) or an active fragment thereof or variants or derivatives thereof as described in WO 2013/170015 and WO 2013/170022. PlySs2, which was identified as an anti-staphylococcal lysin encoded within a prophage of the Streptococcus suis genome, exhibits bacteriocidal and bacteriostatic activity against the bacteria described below in Table 2.

TABLE 2
Reduction in Growth of Different Bacteria and Relative kill
with a lysin, PlySs2 (partial listing).

| Bacteria | Relative Kill with PlySs2 |
|---|---|
| Staphlyococcus aureus | +++ |
| (VRSA, VISA, MRSA, MSSA) | |
| Streptococcus suis | +++ |
| Staphlyococcus epidermidis | ++ |
| Staphlyococcus simulans | +++ |
| Listeria monocytogenes | ++ |
| Enterococcus faecalis | ++ |
| Streptococcus dysgalactiae | ++ |
| Streptococcus agalactiae | +++ |
| Streptococcus pyogenes | +++ |
| Streptococcus equi | ++ |
| Streptococcus sangunis | ++ |
| Streptococcus gordonii | ++ |
| Streptococcus sobrinus | + |
| Streptococcus rattus | + |
| Streptococcus oralis | + |
| Streptococcus pneumoniae | + |
| Bacillus thuringiensis | − |
| Bacillus cereus | − |
| Bacillus subtilis | − |
| Bacillus anthracis | − |
| Escherichia coli | − |
| Enterococcus faecium | − |
| Pseudomonas aeruginosa | − |

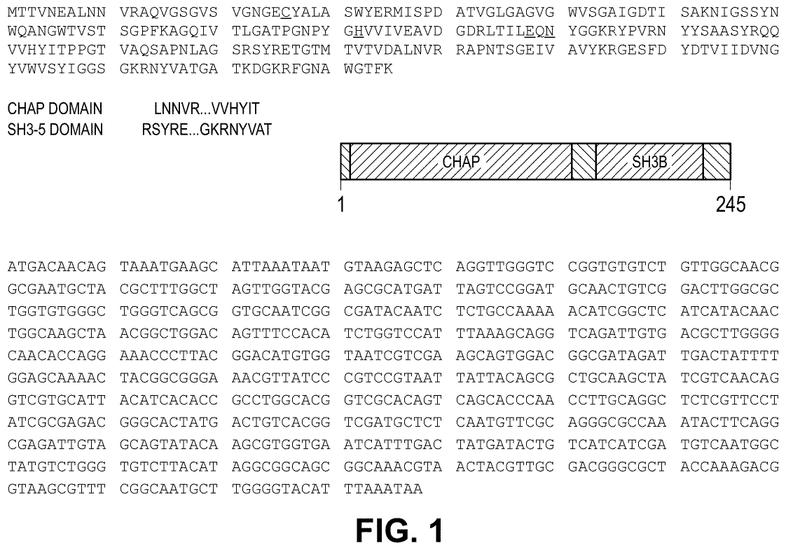

In some embodiments, a lysin suitable for use with the present method is the PlySs2 lysin of SEQ ID NO: 1. The PlySs2 lysin of SEQ ID NO: 1 has a domain arrangement characteristic of most bacteriophage lysins, defined by a catalytic N-terminal domain (FIG. 1) linked to a cell wall-binding C-terminal domain (FIG. 1). The N-terminal domain belongs to the cysteine-histidine-dependent amidohydrolases/peptidases (CHAP) family common among lysins and other bacterial cell wall-modifying enzymes. The C-terminal domain belongs to the SH3b family that often forms the cell wall-binding element of lysins. FIG. 1 depicts the PlySs2 lysin of SEQ ID NO: 1 with the N- and C-terminal domains shown as shaded regions. The N-terminal CHAP domain corresponds to the first shaded amino acid sequence region starting with LNN and the C-terminal SH3b domain corresponds to the second shaded region starting with RSY.

In some embodiments, a lysin suitable for use with the methods disclosed herein comprises one or more of the following lysins: pp55 (SEQ ID NO: 3), pp61 (SEQ ID NO: 4), pp65 (SEQ ID NO: 5), pp296 (SEQ ID NO: 6), pp324 (SEQ ID NO: 7), pp325 (SEQ ID NO: 8), pp338 (SEQ ID NO: 9), pp341 (SEQ ID NO: 10), pp388 (SEQ ID NO: 11), pp400 (SEQ ID NO: 12), pp616 (SEQ ID NO: 13), pp619 (SEQ ID NO: 14), pp628 (SEQ ID NO: 15), pp632 (SEQ ID NO: 16), and pp642 (SEQ ID NO: 17).

In some embodiments, the present methods comprise the administration of a variant lysin to a subject in need thereof. Suitable lysin variants for use with the present method include those polypeptides having at least one substitution, insertion and/or deletion in reference to SEQ ID NO: 1 that retain at least one biological function of the reference lysin. In some embodiments, the variant lysins exhibit antibacterial activity including a bacteriolytic and/or bacteriostatic effect against a broad range of Gram-positive bacteria, including S. aureus and S. epidermidis and an ability to inhibit agglutination, inhibit biofilm formation and/or reduce biofilm. In some embodiments, the present lysin variants render Gram-positive bacteria more susceptible to antibiotics.

In some embodiments, a lysin variant suitable for use with the present methods includes an isolated polypeptide sequence having at least 80%, such as at least 85%, such as at least 90%, such as at least 95%, such as at least 98% or such as at least 99% sequence identity with SEQ ID NO: 1, wherein the variant lysin retains one or more biological activities of the PlySs2lysin having the amino acid sequence of SEQ ID NO: 1 as described herein.

Lysin variants may be formed by any method known in the art and as described in WO 2013/170015, which is herein incorporated by reference in its entirety, e.g., by modifying the PlySs2 lysin of SEQ ID NO: 1 through site-directed mutagenesis or via mutations in hosts that produce the PlySs2 lysin of SEQ ID NO: 1, and which retain one or more of the biological functions as described herein. For example, one of skill in the art can reasonably make and test substitutions or replacements to, e.g., the CHAP domain and/or the SH3b domain of the PlySs2 lysin of SEQ ID NO: 1. Sequence comparisons to the Genbank database can be made with either or both of the CHAP and/or SH3b domain sequences or with the PlySs2 lysin full amino acid sequence of SEQ ID NO: 1, for instance, to identify amino acids for substitution. For example, a mutant or variant having an alanine replaced for valine at valine amino acid residue 19 in the PlySs2 amino acid sequence of SEQ ID NO: 1 is active and capable of killing Gram-positive bacteria in a manner similar to and as effective as the SEQ ID NO: 1 PlySs2 lysin.

Further, as indicated in FIG. 1, the CHAP domain contains conserved cysteine and histidine amino acid sequences (the first cysteine and histidine in the CHAP domain) which are characteristic and conserved in CHAP domains of different polypeptides. It is reasonable to predict, for example, that the conserved cysteine and histidine residues should be maintained in a mutant or variant of PlySs2 so as to maintain activity or capability. Accordingly, particularly desirable residues to retain in a lysin variant of the present disclosure include active-site residues Cys₂₆, His₁₀₂, Glu₁₁₈, and Asn₁₂₀ in the CHAP domain of SEQ ID NO: 1. Particularly desirable substitutions include: Lys for Arg and vice versa such that a positive charge may be maintained, Glu for Asp and vice versa such that a negative charge may be maintained, Ser for Thr such that a free —OH can be maintained and Gln for Asn such that a free NH2 can be maintained.

Suitable variant lysins are described in PCT Published Application No. WO 2019/165454 (International Application No.: PCT/US2019/019638), which is herein incorporated by reference in its entirety. Particularly, suitable variant lysins include those set forth herein as SEQ ID NOS: 3-17 as well as variant lysins having at least 80%, such as at least 85%, such as at least 90%, such as at least 95%, such as at least 98% or such as at least 99% sequence identity with any one of SEQ ID NOS: 3-17, wherein the variant lysin retains one or more biological activities of the PlySs2 lysin having the amino acid sequence of SEQ ID NO: 1 as described herein.

SEQ ID NOs: 3-17 are modified lysin polypeptides having at least one amino acid substitution relative to a counterpart wild-type PlySs2 lysin SEQ ID NO: 1, while preserving antibacterial activity and effectiveness. SEQ ID NOs: 3-17 may be described by reference to their amino acid substitutions with respect to SEQ ID NO: 1, as shown below in Table A. The amino acid sequences of the modified lysin polypeptides (referencing differences from SEQ ID NO: 1 and the positions of its amino acid residues) are summarized using one-letter amino acid codes as follows:

TABLE A

| | Substitution location | | | | | | | |
|---|---|---|---|---|---|---|---|---|
| No. | TCE 1 | TCE 2 | TCE 3 | TCE 4 | TCE 5 | TCE 6 | TCE 7 | TCE 8 |
| pp55 (SEQ ID NO: 3) | | L92W | V104S | V128T | | | | |
| | | | | and | | | | |
| | | | | Y137S | | | | |
| pp61 (SEQ ID NO: 4) | | L92W | V104S | V128T | | | S198H | I206E |
| | | | | and | | | | |
| | | | | Y137S | | | | |
| pp65 (SEQ ID NO: 5) | | L92W | V104S | V128T | | | S198Q | V204A |
| | | | | and | | | | and |
| | | | | Y137S | | | | V212A |
| pp296 (SEQ ID NO: 6) | | L92W | V104S | V128T | Y164K | N184D | S198Q | |
| | | | | and | | | | |
| | | | | Y137S | | | | |
| pp324 (SEQ ID NO: 7) | | L92W | V104S | V128T | Y164N | N184D | | |
| | | | | and | | | | |
| | | | | Y137S | | | | |
| pp325 (SEQ ID NO: 8) | | L92W | V104S | V128T | Y164N | | R195E | |
| | | | | and | | | | |
| | | | | Y137S | | | | |
| pp338 (SEQ ID NO: 9) | | L92W | V104S | V128T | | N184D | S198H | |
| | | | | and | | | | |
| | | | | Y137S | | | | |
| pp341 (SEQ ID NO: 10) | | L92W | V104S | V128T | | N184D | | V204A |
| | | | | and | | | | and |
| | | | | Y137S | | | | V212A |
| pp388 (SEQ ID NO: 11) | | | | | Y164N | N184D | R195E | V204K |
| | | | | | | | | and |
| | | | | | | | | V212E |
| pp400 (SEQ ID NO: 12) | R35E | L92W | V104S | V128T | | | | |
| | | | | and | | | | |
| | | | | Y137S | | | | |
| pp616 (SEQ ID NO: 13) | | | | V1281 | Y164K | | | |
| | | | | and | | | | |
| | | | | Y137S | | | | |
| pp619 (SEQ ID NO: 14) | | L92W | V104S | V128T | Y164K | | | |
| | | | | and | | | | |
| | | | | Y137S | | | | |
| pp628 (SEQ ID NO: 15) | | L92W | V104S | V128T | Y164K | | | V204K |
| | | | | and | | | | and |
| | | | | Y137S | | | | V212E |
| pp632 (SEQ ID NO: 16) | | L92W | V104S | V128T | Y164K | N184D | S198Q | V204K |
| | | | | and | | | | and |
| | | | | Y137S | | | | V212E |
| pp642 (SEQ ID NO: 17) | | L92W | V104S | V128T | Y164K | | | I206E |
| | | | | and | | | | and |
| | | | | Y137S | | | | V214G |

In some embodiments the present method includes administering an active fragment of a lysin to a subject in need thereof. Suitable active fragments include those that retain a biologically active portion of a protein or peptide fragment of the lysin embodiments, as described herein. Such variants include polypeptides comprising amino acid sequences that include fewer amino acids than the full length protein of the lysin protein and exhibit at least one activity of the corresponding full-length protein. Typically, biologically active portions comprise a domain or motif with at least one activity of the corresponding protein. An exemplary domain sequence for the N-terminal CHAP domain of the PlySs2 lysin is provided in FIG. 1. An exemplary domain sequence for the C terminal SH3b domain of the PlySs2lysin is also provided in FIG. 1. A biologically active portion of a protein or protein fragment of the disclosure can be a polypeptide which is, for example, 10, 25, 50, 100 amino acids in length. Other biologically active portions, in which other regions of the protein are deleted can be prepared by recombinant techniques and evaluated for one or more of the functional activities of the native form of a polypeptide of the embodiments.

In some embodiments, suitable active fragments include those having at least 80%, such as at least 85%, such as at least 90%, such as at least 95%, such as at least 98% or such as at least 99% sequence identity with the active fragments described herein, wherein the active fragment thereof retains at least one activity of a CHAP and/or the SH3b domain, e.g., as shown in FIG. 1.

A lysin or active fragment thereof or variant or derivative thereof as described herein for use in the present method may be produced by a bacterial organism after being infected with a particular bacteriophage or may be produced or prepared recombinantly or synthetically. In as much the lysin polypeptide sequences and nucleic acids encoding the lysin polypeptides are described and referenced herein, the present lysins may be produced via the isolated gene for the lysin from the phage genome, putting the gene into a transfer vector, and cloning said transfer vector into an expression system, using standard methods of the art, as described for example in WO 2013/170015, which is herein incorporated by reference in its entirety. The present lysin variants may be truncated, chimeric, shuffled or “natural,” and may be in combination as described, for example, in U.S. Pat. No. 5,604,109, which is incorporated herein in its entirety by reference.

Mutations can be made in the amino acid sequences, or in nucleic acid sequences encoding the polypeptides and lysins described herein, including in the lysin sequence set forth in SEQ ID NO: 1, or in active fragments or truncations thereof, such that a particular codon is changed to a codon which codes for a different amino acid, an amino acid is substituted for another amino acid, or one or more amino acids are deleted.

Such a mutation is generally made by making the fewest nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present disclosure should be considered to include sequences containing conservative changes which do not significantly alter the activity or binding characteristics of the resulting protein. Thus, one of skill in the art, based on a review of the sequence of the PlySs2 lysin polypeptide provided herein and on their knowledge and the public information available for other lysin polypeptides, can make amino acid changes or substitutions in the lysin polypeptide sequence. Amino acid changes can be made to replace or substitute one or more, one or a few, one or several, one to five, one to ten, or such other number of amino acids in the sequence of the lysin(s) provided herein to generate mutants or variants thereof. Such mutants or variants thereof may be predicted for function or tested for function or capability for anti-bacterial activity as described herein against, e.g., Staphylococcal, Streptococcal, or Enterococcal bacteria, and/or for having comparable activity to the lysin(s) as described and particularly provided herein. Thus, changes made to the sequence of lysin, and mutants or variants described herein can be tested using the assays and methods known in the art and described herein. One of skill in the art, on the basis of the domain structure of the lysin(s) hereof can predict one or more, one or several amino acids suitable for substitution or replacement and/or one or more amino acids which are not suitable for substitution or replacement, including reasonable conservative or non-conservative substitutions.

Antibiotics

In some embodiments, the methods of treating or preventing bone and joint infections as described herein comprise co-administering a therapeutically effect amount of one or more antibiotics and a PlySs2 lysin. In some embodiments, co-administration of a lysin or active fragment thereof or variant or derivative thereof and one or more antibiotic as described herein results in a synergistic bacteriocidal and/or bacteriostatic effect on Gram-positive bacteria such as S. aureus or S. epidermidis. Typically, the co-administration results in a synergistic effect on bacteriostatic and/or bactericidal activity. In other embodiments, the co-administration is used to suppress virulence phenotypes including biofilm formation and/or agglutination. In some embodiments, the co-administration is used to reduce an amount of biofilm in a subject.

Suitable antibiotics for use with the present methods include antibiotics of different types and classes, such as beta-lactams including penicillins (e.g. methicillin, oxacillin), cephalosporins (e.g. cefalexin and cefactor), monobactams (e.g. aztreonam) and carbapenems (e.g. imipenem and entapenem); macrolides (e.g. erythromycin, azithromycin), aminoglycosides (e.g. gentamicin, tobramycin, amikacin), glycopeptides (e.g., vancomycin, teicoplanin), oxazolidinones (e.g linezolid and tedizolid), lipopeptides (e.g. daptomycin) and sulfonamides (e.g. sulfamethoxazole).

In some embodiments, the antibiotics comprise a rifamycin antibiotic, such as rifampin or rifabutin. Typically, a rifamcyin antibiotic is used.

In some embodiments, the antibiotic is an antibiotic typically used to treat osteomyelitis, such as acute osteomyelitis, such as vancomycin or daptomycin. In some embodiments, the antibiotic penetrates bone tissue well, e.g. daptomycin.

Additional Methods of the Disclosure

In another aspect, the present disclosure is directed to a method of preventing a bone or joint infection due to a Gram-positive bacteria as described herein, which method comprises: administering a therapeutically effective amount of a PlySs2 lysin or variant thereof as described herein to a subject in need thereof. Optionally, an antibiotic as described herein is co-administered with the PlySs2 lysin.

In some embodiments, the PlySs2 lysin or variant thereof as herein described is administered in conjunction with Debridement and Implant Retention (DAIR). In these embodiments, debridement of infected and potentially infected tissues around e.g., an implant, is typically performed followed by arthroscopic irrigation of involved tissues with copious volumes of fluid, such as sterile saline. In some embodiments, a PlySs2 lysin or variant thereof as described herein is administered during arthroscopy, before, during or after arthroscopic irrigation. In some embodiments, conventional antibiotics as described herein, such as tedizolid, are subsequently orally or intravenously administered to the subject for e.g., 6-24 weeks.

In some embodiments, the subject to be administered a lysin of the disclosure is elderly or suffers from a condition associated with a higher risk of a bone or joint infection. For example, the subject at risk for a bone or joint infection may suffer from obesity, e.g., a body mass index (BMI) threshold of 35. An elderly subject, for example, is at least 65 years, such as 65-90 years, 75-90 years, or 79-89 years. Without being limited by theory, possible reasons for the increased risk of bone or joint infections, such as prosthetic bone or joint infections, with obesity include prolonged operative duration and/or the presence of other comorbidities.

In some embodiments, the subject at risk for a bone or joint infection, particularly a prosthetic joint infection, suffers from diabetes mellitus. Without being limited by theory, the risk associated with diabetes may be due to increased biofilm formation in the presence of elevated levels of glucose, impaired leukocyte function, or microvascular changes in subjects with diabetes mellitus, which may influence wound healing and the development of superficial surgical site infections.

Other risk factors for bone and/or joint infections include rheumatoid arthritis, male gender and smoking. In addition, a diagnosis of bacteremia in the year preceding an implant surgery is also a risk factor for a bone and/or joint infection, such as a prosthetic joint infection.

In another aspect, the present disclosure is directed to a method for inhibiting the formation of a Gram-positive bacterial biofilm or disrupting a Gram-positive bacterial biofilm formed in a synovial fluid comprising administering a composition comprising a lysin capable of killing a Gram-positive bacteria as herein described, wherein the lysin is a PlySs2 lysin as also described herein and the biofilm is effectively inhibited or dispersed. The Gram-positive bacteria in this aspect of the disclosure may include any of the Gram-positive bacteria described herein. However, typically, the Gram-positive bacteria is Staphylococcus epidermidis.

Dosages and Administration

Dosages of the present lysins or active fragments thereof or variants or derivatives thereof that are administered to a subject in need thereof depend on a number of factors including the activity of infection being treated, the age, health and general physical condition of the subject to be treated, the activity of a particular lysin or active fragment thereof or variant or derivative thereof, the nature and activity of the antibiotic, if any, with which a lysin or active fragment thereof or variant or derivative thereof according to the present disclosure is being paired and the combined effect of such pairing. Generally, effective amounts of the present lysins or active fragments thereof or variants or derivatives thereof to be administered are anticipated to fall within the range of 0.00001-200 mg/kg, such as 0.2 mg/kg to about 0.3 mg/kg, such as 0.25 mg/kg, such as, 1-150 mg/kg, such as 40 mg/kg to 100 mg/kg and are administered 1-4 times daily for a period up to 14 days. The antibiotic may be administered at standard dosing regimens or in lower amounts in view of e.g., synergy. All such dosages and regimens however (whether of the lysin or active fragment thereof or variant or derivative thereof or any antibiotic administered in conjunction therewith) are subject to optimization. Optimal dosages can be determined by performing in vitro and in vivo pilot efficacy experiments as is within the skill of the art but taking the present disclosure into account.

It is contemplated that the present lysins or active fragments thereof or variants or derivatives thereof provide a bactericidal and, when used in smaller amounts, a bacteriostatic effect, and are active against a range of antibiotic-resistant bacteria and are not associated with evolving resistance. Based on the present disclosure, in a clinical setting, the present lysins or active fragments thereof or variants or derivatives thereof are a potent alternative (or additive or component) of compositions for treating bone and joint infections arising from drug- and multidrug-resistant bacteria when combined with certain antibiotics (even antibiotics to which resistance has developed). Existing resistance mechanisms for Gram-positive bacteria should not affect sensitivity to the lytic activity of the present polypeptides.

For any polypeptide of the present disclosure, the therapeutically effective dose can be estimated initially either in cell culture assays or in animal models, usually mice, rabbits, dogs, or pigs. The animal model can also be used to achieve a desirable concentration range and route of administration. Obtained information can then be used to determine the effective doses, as well as routes of administration in humans. However, typically systemic administration, in particular intravenous administration, is used. Dosage and administration can be further adjusted to provide sufficient levels of the active ingredient or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy and the judgment of the treating physician.

A treatment regimen can entail daily administration (e.g., once, twice, thrice, etc. daily), every other day (e.g., once, twice, thrice, etc. every other day), semi-weekly, weekly, once every two weeks, once a month, etc. In one embodiment, treatment can be given as a continuous infusion. Unit doses can be administered on multiple occasions. Intervals can also be irregular as indicated by monitoring clinical symptoms. Alternatively, the unit dose can be administered as a sustained release formulation, in which case less frequent administration is required. Dosage and frequency may vary depending on the patient. It will be understood by one of skill in the art that such guidelines will be adjusted for localized administration, e.g. intranasal, inhalation, rectal, etc., or for systemic administration, e.g. oral, rectal (e.g., via enema), i.m. (intramuscular), i.p. (intraperitoneal), i.v. (intravenous), s.c. (subcutaneous), transurethral, and the like.

In some embodiments, the present lysins or active fragments thereof or variants or derivatives thereof and one or more antibiotics as described herein, such as daptomycin, are administered simultaneously. In other embodiments, the present lysins or active fragments thereof or variants or derivatives thereof and the one or more antibiotics of the present method, such as daptomycin, are administered in series, such as sequentially, in any order. In some embodiments, the lysin is administered during or subsequent to administration of a standard of care antibiotic treatment, e.g., a two-week course of oxacillin and gentamicin or daptomycin. The present lysins or active fragments thereof or variants or derivatives thereof and the present one or more antibiotics may be administered in a single dose or multiple doses, singly or in combination.

The lysins or active fragments thereof or variants or derivatives thereof and the one or more antibiotics of the present disclosure may be administered by the same mode of administration or by different modes of administration, and may be administered once, twice or multiple times, one or more in combination or individually. Thus, the present lysins or active fragments thereof or variants or derivatives thereof may be administered in an initial dose followed by a subsequent dose or doses, particularly depending on the response, e.g., the bacteriocidal and/or bacteriostatic effects and/or the effect on agglutination and/or biofilm formation or reduction, and may be combined or alternated with antibiotic dose(s). Typically, the lysins or active fragments thereof or variants or derivatives thereof are administered in a single bolus followed by conventional doses and administration modes of the one or more antibiotics of the present disclosure.

In more typical embodiments, a single bolus of a lysin or active fragment thereof or variant or derivative thereof of the present disclosure is administered to a subject followed by a conventional regimen, e.g., standard of care (SOC) dosages, of one or more antibiotics of the present disclosure, such as daptomycin. In other typical embodiments, one or more antibiotics of the present disclosure, such as daptomycin, is administered to a subject followed by a single bolus of a lysin or active fragment thereof or variant or derivative thereof of the present disclosure, followed by additional dosages of the one or more antibiotics of the present disclosure at conventional dosages, such as daptomycin.

In some embodiments, the lysins or active fragments thereof or variants or derivatives thereof may be administered at sub-MIC levels, e.g., at sub-MIC levels ranging from 0.9×MIC to 0.0001×MIC. At such sub-MIC levels, the present lysins or active fragments thereof or variants or derivatives thereof are typically used to inhibit the growth of Gram-positive bacteria, reduce agglutination, and/or inhibit biofilm formation or to reduce or eradicate biofilm.

In some embodiments, a single sub-MIC dose of the lysin or active fragment thereof or variant or derivative thereof is administered to a subject followed by a conventional regimen of one or more doses of the one or more antibiotics of the present disclosure. In other, even more typical embodiments, one or more antibiotics of the present disclosure such as daptomycin is administered to a subject at a conventional dosage followed by a single bolus at a sub-MIC dose of lysin or active fragment thereof or variant or derivative thereof of the present disclosure, followed by additional dosages of the one or more antibiotics of the present disclosure at conventional dosages, such as daptomycin.

Without being limited by theory, sub-MIC dosages of the present lysins or active fragments thereof or variants or derivatives thereof can result in non-lethal damage to the cell envelope, mediated by peptidoglycan hydrolytic activity of the lysins or active fragments thereof or variants or derivatives thereof. In some embodiments, the resulting physical and functional changes in the cell envelope account for growth delays. Such physical and functional changes include e.g., destabilization of the cell wall, increases in membrane permeability and dissipation of membrane potential. Although the present lysins or active fragments thereof or variants or derivatives thereof do not, typically, directly act on the bacterial cell membrane, any effects on cell membrane permeability and electrostatic potential are likely the result of osmotic stress induced by the peptidoglycan hydrolytic activity of lysin (and destabilization of the cell envelope) at very low concentrations. It is also postulated that localized cell wall hydrolysis can result in the extrusion of inner membrane and the formation of pores as well as the uncoupling of cell synthesis and hydrolysis, changes in cell wall thickness resulting in subsequent growth arrest.

In some embodiments, the sub-MIC concentrations of the present lysins or active fragments thereof or variants or derivatives thereof damage the bacterial cell envelope resulting in bacteria that are more susceptible to conventional antibiotics than in the absence of the sub-MIC dose of the present lysins or active fragments thereof or variants or derivatives thereof.

In some embodiments, the present lysin or active fragment thereof or variant or derivative thereof at sub-MIC and/or MIC level doses are capable of reducing a biofilm, in particular an in vivo biofilm. As is known in the art, in vivo biofilms may be structurally distinct from in vitro biofilms. Typically, the reason for the differences between in vitro biofilms and in vivo biofilms, such as those associated with chronic infections, is the lack of defense mechanism exposure in in vitro biofilm systems. In most in vivo biofilm habitats, phagocytes, and even bacteriophages may be present, along with the presence of pus and other excreted fluids and polymers. Such variables are generally avoided in in vitro model systems where they may be difficult to control or reproduce.

In some embodiments, the one or more antibiotics of the present disclosure are administered to a subject in need thereof at the MIC level or greater than the MIC level, such as 1×MIC, 2×MIC, 3×MIC and 4×MIC. In other embodiments, the antibiotics are administered at a sub-MIC level, e.g., ranging from 0.9×MIC to 0.0001×MIC.

In some embodiments, a single sub-MIC dose of the lysin or active fragment thereof or variant or derivative thereof of the present disclosure is administered to a subject followed by one or more doses of the one or more antibiotics of the present disclosure, such as daptomycin, wherein the antibiotic dose(s) is also administered at a sub-MIC level.

In other embodiments, one or more antibiotics of the present disclosure such as daptomycin is administered to a subject at a sub-MIC dosage followed by a single bolus at a sub-MIC dosage of a lysin or active fragment thereof or variant or derivative thereof of the present disclosure, followed by one or more additional dosages of the one or more antibiotics of the present disclosure at sub-MIC dosages, such as daptomycin.

Formulations

The lysin or active fragment thereof or variant or derivatives thereof of the present disclosure, optionally administered either alone or in combination or in series, with the one or more antibiotics described herein may each be included in a single pharmaceutical formulation or be separately formulated in the form of a solution, a suspension, an emulsion, an inhalable powder, an aerosol, or a spray, tablets, pills, pellets, capsules, capsules containing liquids, powders, sustained-release formulations, suppositories, tampon applications emulsions, aerosols, sprays, suspensions, lozenges, troches, candies, injectants, chewing gums, ointments, smears, time-release patches, liquid absorbed wipes, and combinations thereof.

In some embodiments, administration of the pharmaceutical formulations may include systemic administration. Systemic administration can be enteral or oral, i.e., a substance is given via the digestive tract, parenteral, i.e., a substance is given by other routes than the digestive tract such as by injection or inhalation. Thus, the lysins or active fragments thereof or variants or derivatives thereof and optionally the one or more antibiotics of the present disclosure can be administered to a subject orally, parenterally, by inhalation, topically, rectally, nasally, buccally or via an implanted reservoir or by any other known method. The lysins or active fragments thereof or variants or derivatives thereof and/or the one or more antibiotics of the present disclosure can also be administered by means of sustained release dosage forms.

For oral administration, the lysins or active fragments thereof or variants or derivatives thereof and optionally, the one or more antibiotics of the present disclosure can be formulated into solid or liquid preparations, for example tablets, capsules, powders, solutions, suspensions and dispersions. In some embodiments, the lysins or active fragments thereof or variants or derivatives thereof and/or the one or more antibiotics of the present disclosure can be formulated with excipients such as, e.g., lactose, sucrose, corn starch, gelatin, potato starch, alginic acid and/or magnesium stearate.

For preparing solid compositions such as tablets and pills, the lysins or active fragments thereof or variants or derivatives thereof and/or the one or more antibiotics of the present disclosure is mixed with a pharmaceutical excipient to form a solid pre-formulation composition. If desired, tablets may be sugar coated or enteric coated by standard techniques. The tablets or pills may be coated or otherwise compounded to provide a dosage form affording the advantage of prolonged action. For example, the tablet or pill can include an inner dosage and an outer dosage component, the latter being in the form of an envelope over the former. The two dosage components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In another embodiment, the pharmaceutical formulations of the present disclosure are formulated as inhalable compositions. In some embodiments, the present pharmaceutical formulations are advantageously formulated as a dry, inhalable powder. In specific embodiments, the present pharmaceutical formulations may further be formulated with a propellant for aerosol delivery. Examples of suitable propellants include, but are not limited to: dichlorodifluoromethane, trichlorofluoromethane, dichloro-tetrafluoroethane and carbon dioxide. In certain embodiments, the formulations may be nebulized.

In some embodiments, the inhalable pharmaceutical formulations include excipients. Examples of suitable excipients include, but are not limited to: lactose, starch, propylene glycol diesters of medium chain fatty acids; triglyceride esters of medium chain fatty acids, short chains, or long chains, or any combination thereof; perfluorodimethylcyclobutane; perfluorocyclobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

A surfactant can be added to an inhalable pharmaceutical formulation of the present disclosure in order to lower the surface and interfacial tension between the medicaments and the propellant. The surfactant may be any suitable, non-toxic compound which is non-reactive with the present polypeptides. Examples of suitable surfactants include, but are not limited to: oleic acid; sorbitan trioleate; cetyl pyridinium chloride; soya lecithin; polyoxyethylene(20) sorbitan monolaurate; polyoxyethylene (10) stearyl ether; polyoxyethylene (2) oleyl ether; polyoxypropylene-polyoxyethylene ethylene diamine block copolymers; polyoxyethylene(20) sorbitan monostearate; polyoxyethylene(20) sorbitan monooleate; polyoxypropylene-polyoxyethylene block copolymers; castor oil ethoxylate; and combinations thereof.

In some embodiments, the pharmaceutical formulations of the present disclosure comprise nasal formulations. Nasal formulations include, for instance, nasal sprays, nasal drops, nasal ointments, nasal washes, nasal injections, nasal packings, bronchial sprays and inhalers, or indirectly through use of throat lozenges, mouthwashes or gargles, or through the use of ointments applied to the nasal nares, or the face or any combination of these and similar methods of application.

The pharmaceutical formulations of the present disclosure are more typically administered by injection. For example, the pharmaceutical formulations can be administered intramuscularly, intrathecally, subdermally, subcutaneously, or intravenously to treat infections by Gram-positive bacteria, typically, bone or joint infections caused by S. epidermidis. The pharmaceutically acceptable carrier may be comprised of distilled water, a saline solution, albumin, a serum, or any combinations thereof. Additionally, pharmaceutical formulations of parenteral injections can comprise pH buffered solutions, adjuvants (e.g., preservatives, wetting agents, emulsifying agents, and dispersing agents), liposomal formulations, nanoparticles, dispersions, suspensions or emulsions as well as sterile powders for reconstitution into sterile injectable solutions or dispersions just prior to use.

In cases where parenteral injection is the chosen mode of administration, an isotonic formulation is typically used. Generally, additives for isotonicity can include sodium chloride, dextrose, mannitol, sorbitol, and lactose. In some cases, isotonic solutions such as phosphate buffered saline are preferred. Stabilizers can include gelatin and albumin. A vasoconstriction agent can be added to the formulation. The pharmaceutical preparations according to this type of application are provided sterile and pyrogen free.

The pharmaceutical formulations of the present disclosure may be presented in unit dosage form and may be prepared by any methods well known in the art. The amount of active ingredients which can be combined with a carrier material to produce a single dosage form will vary depending upon the host being treated, the duration of exposure of the recipient to the infectious bacteria, the size and weight of the subject, and the particular mode of administration. The amount of active ingredients that can be combined with a carrier material to produce a single dosage form will generally be that amount of each compound which produces a therapeutic effect. Generally, out of one hundred percent, the total amount will range from about 1 percent to about ninety-nine percent of active ingredients, typically from about 5 percent to about 70 percent, most typically from about 10 percent to about 30 percent.

### EXAMPLES

### Example 1. Antimicrobial Activity of CF-301 Against S. epidermidis in Human Synovial Fluid (HSF)

Minimum Inhibitory Concentrations (MICs) of the CF-301 lysin (SEQ ID NO: 1) against S. epidermidis were determined using broth microdilution (BMD) following CLSI methodology (M07-A11, 2018) in a non-standard medium (caMHB supplemented to 25% with horse serum and 0.5 mM with DTT (caMHB-HSD)) approved for use by the CLSI in antimicrobial susceptibility testing with CF-301 (CLSI, AST Subcommittee Meeting, January, 2018). Activity of CF-301 against S. epidermidis in HSF (Discovery Life Sciences) was similarly determined using BMD in caMHB with 50% HSF (caMHB-HSF). The caMHB-HSF supports the growth and biofilm formation of S. epidermidis as well as S. aureus. Fifty-three S. epidermidis clinical isolates and two MRSA strains were chosen for study; each isolate was previously demonstrated to form biofilms in a previous study (Schuch et al. (2017) AAC, 61:e02666-16).

As shown in Table 3, below, CF-301 demonstrated potent activity against S. epidermidis in human synovial fluid with a MIC_{50/90} of 0.015/0.125 μg/mL and a range of 0.0078-2 μg/mL. As also indicated in Table 3, CF-301 activity against S. epidermidis was similar to that observed against S. aureus.

TABLE 3
Antimicrobial Activity of Exebacase Against S. epidermidis

| | CF-301 MIC (μg/mL) | | | | | |
|---|---|---|---|---|---|---|
| Organism | caMHB-HSF | | | caMHB-HSD | | |
| (# of isolates) | MIC₅₀ | MIC₉₀ | Range | MIC₅₀ | MIC₉₀ | Range |
| S. | 0.015 | 0.125 | 0.0078-2 | 0.5 | 0.5 | 0.25-2 |
| epidermidis | | | | | | |
| (N = 53) | | | | | | |

| S. aureus | CF-301 MIC (μg/mL) | |
|---|---|---|
| strain | caMHB-HSF | caMHB-HSD |
| MW2 | 0.03 | 0.5 |
| ATCC | 0.03 | 0.5 |
| BAA-42 | | |

### Example 2. Disruption of S. epidermidis Biofilms by CF-301 in HSF

Macroscopic analysis of CF-301 activity on biofilms, which were formed in human synovial fluid, was performed in the manner described in Dastgheyb et al. (2015) JID 211:641-50 and Dastgheyb et al. (2015) AAC 59:e04579-14. Briefly, 10⁸ CFUs of S. epidermidis isolate NRS6 were incubated for 24 hours at 37° C. in 24-well plates containing HSF. After biofilm formation, the wells were stained with ethidium bromide (EtBr) and treated with 0.1 or 1 μg/mL CF-301 for 2 hours. The biofilms were visualized by UV fluorescent imaging. Untreated controls were also examined.

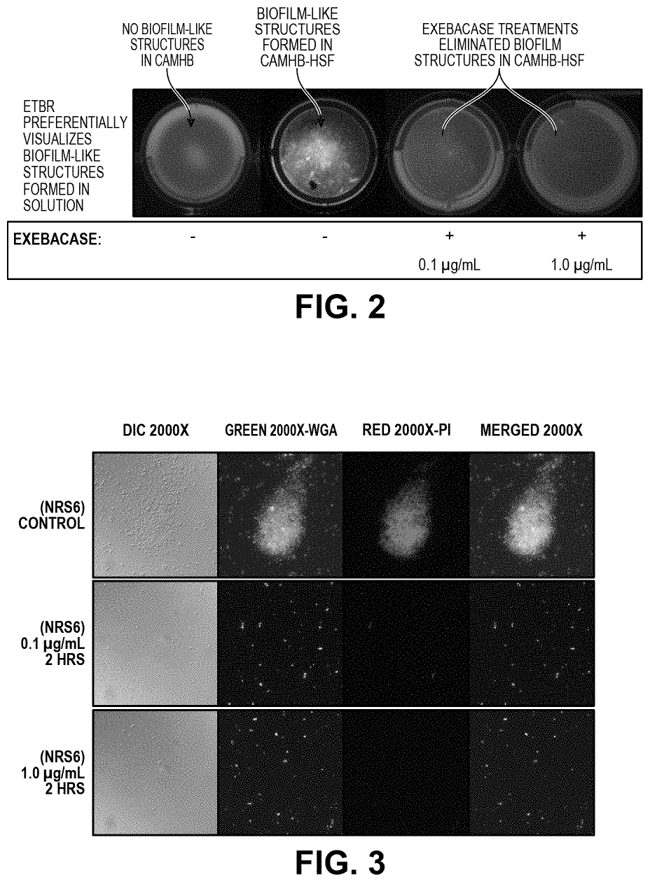

FIG. 2 shows the impact of CF-301 treatment on ethidium bromide staining of biofilm structures formed by NRS6 in human synovial fluid. As evidenced in FIG. 2, the biofilm structures were eliminated within 2 hours.

The biofilms were also stained with Alexa Flour⁴⁸⁸-WGA, which stained the exopolysaccharide in the biofilms (and individual bacteria) and propidium iodide (PI) which stained the entire biofilm. The biofilms were then visualized by fluorescence microscopy. As evidenced in FIG. 3, the S. epidermidis biofilms formed in HSF were eliminated after two hours of treatment with 0.1 ug/mL or 1 ug/mL CF-301.

### Example 3. SEM Analysis of Biofilm Disruption in HSF

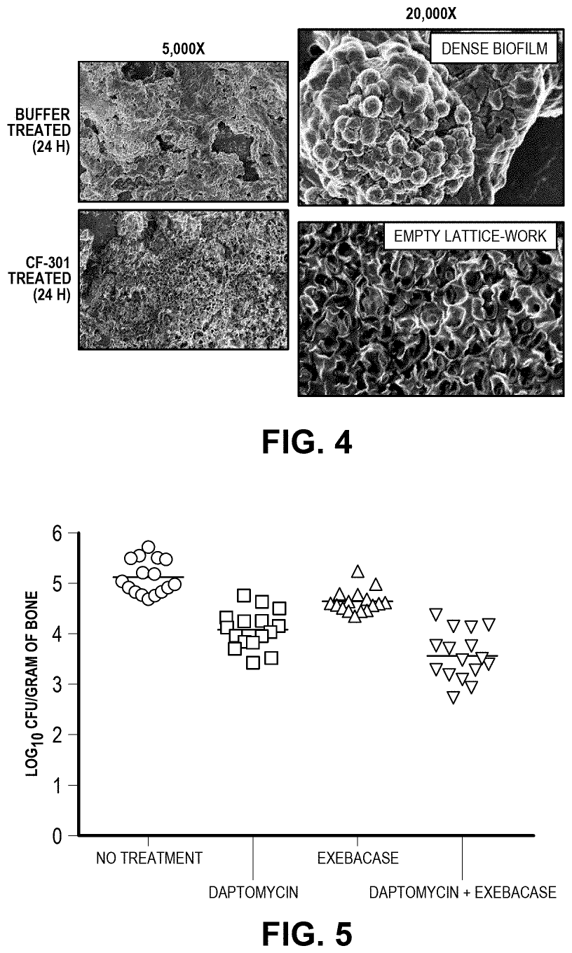

Scanning electron microscopy (SEM) was also used to evidence biofilm formation by S. aureus in human synovial fluid and elimination after 2 hour treatments with CF-301 at concentrations of 0.01, 0.1 and 1 μg/mL. In this example, S. aureus biofilms were formed in human synovial fluid before CF-301 treatment. As shown in FIG. 4, CF-301 disrupts these biofilms.

The foregoing examples support that lysins, such as CF-301, may be used as a treatment for bone and joint infections, particularly prosthetic joint infections, including those caused by S. epidermidis, which are complicated by biofilms against which antibiotics are generally poorly effective.

### Example 4. Exebacase (CF-301) Combined with Daptomycin is More Active than Daptomycin or CF-301 Alone in Methicillin-Resistant Staphylococcus aureus Osteomyelitis in Rats

Levels of CF-301 in bone were found to be about 10-15% that of plasma levels after a single dose of 10 mg/kg, thereby offering a strategy to target bone and joint infections and lyse S. aureus causing infection at such sites. In order to test the efficacy of CF-301 against bone infections, an animal model of acute MRSA osteomyelitis was used. The strain used to establish infection, MRSA IDRL-6169, had minimum inhibitory concentrations of 0.5 μg/ml for both CF-301 and daptomycin, as determined by broth microdilution. Minimum biofilm inhibitory concentrations and minimum biofilm bactericidal concentration were 1 and 4 μg/ml for CF-301 and 1 and 2 μg/ml for daptomycin, respectively, as determined using previously described methods. See Schmidt-Malan et al., 2016, Diag. Microbiol. Infect. Dis. 85:77-79. All CF-301 testing was supplemented with 0.5 mM DL-dithiothreitol and 25% horse serum as described in Schuch R. 2016, Methods Development and Standardization Working Group, Clinical Laboratory Science Institute, Wayne, Pa.

Acute osteomyelitis was established in 64 male Sprague Dawley rats using a modification of Zak's model of experimental osteomyelitis O'Reilly T et al. 1999. “Rat model of bacterial osteomyelitis of the tibia, p 561-575.” In Zak O, Sande M (ed), Handbook of animal models of infection. Academic Press, San Diego, Calif. Animals were anesthetized with isoflurane and the left knee was shaved and disinfected with chlorohexidine. To induce osteomyelitis, the knee joint was bent at a 45 degree angle to expose the top of the tibial process. A 1 milliliter syringe with a 21 gauge needle containing 10 μl arachidonic acid (50 μg/ml) and 50 μl of a 10⁷ cfu suspension of MRSA IDRL-6169 in tryptic soy broth was inserted into the tibia. The bacterial suspension was slowly injected into the tibia, the needle removed, the knee joint straightened and pressure placed on the injection site for 1 minute.

One week after establishing infection (Day 8), rats were randomly assigned to one of four treatment arms: 1) no treatment, 2) 60 mg/kg daptomycin intraperitoneally every 12 hours for four days, 3) single dose 40 mg/kg CF-301 in the tail vein or 4) single dose 40 mg/kg CF-301 plus 60 mg/kg daptomycin intraperitoneally every 12 hours for four days. Daptomycin was administered 15 minutes prior to CF-301 injection. CF-301 was maintained on ice until injection. Rats were sacrificed 4 days after the start of therapy (Day 12). The left tibia from each animal was collected, weighed and cryopulverized for quantitative bacterial culture. Results of quantitative cultures were compared using SAS software version 9.4 (SAS Inc., Cary, N.C.) using the Kruskal-Wallis test. Means and standard deviation were reported as log₁₀ colony forming units (cfu)/gram of bone. All tests were two sided; p-values less than 0.05 were considered statistically significant.

Results

Rats receiving no treatment had a mean (±SD) bacterial density of 5.13 (±0.34) log₁₀ cfu/gram of bone. Rats in the daptomycin, CF-301 and daptomycin plus CF-301 therapy groups had means (±SDs) of 4.09 (±0.37), 4.65 (±0.65) and 3.57 (±0.48) log₁₀ cfu/gram of bone, respectively (FIG. 5). Compared to untreated rats, there were reductions of 1.04, 0.65 and 1.56 log₁₀ cfu/gram of bone with daptomycin, CF-301 and CF-301 plus daptomycin therapy, respectively. Colony counts in all treatment groups were significantly reduced compared to untreated rats (P<0.0001). However, daptomycin with CF-301 animals had lower colony counts than did those treated with daptomycin (P=0.0042) or exebacase (P<0.0001) alone.

The above-described results support that CF-301, alone or in combination with an antibiotic, such as daptomycin, may be used to treat osteomyelitis. While treatment with daptomycin or CF-301 alone showed a reduction in infection, CF-301 and daptomycin combined showed a better effect.

### Example 5. Efficacy of CF-301 During Arthroscopic DAIR in Patients with Prosthetic Knee Infection

Elderly patients (79 to 89 years) with recurrent multiple drug resistant (MDR) Staphylococcus epidermidis prosthetic knee infection for whom revision or transfemoral amputation was not feasible and for whom no other oral option was available, were identified for treatment with CF-301 in combination with DAIR. Each case was discussed with the French Health Authority in accordance with the local ethics committee. Prior to treatment, each patient signed a written consent. CF-301 (75 mg/mL; 30 mL) was directly administered into the joint during arthroscopy followed by suppressive tedizolid as salvage therapy.

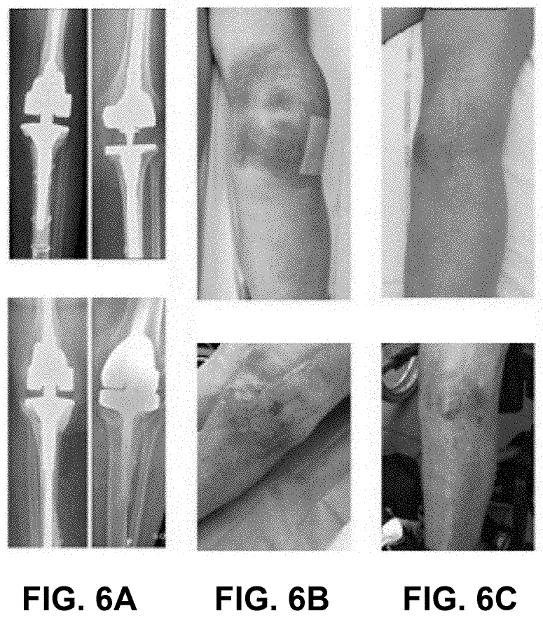

Four patients were treated. All had several previous prosthetic knee revisions without prosthesis loosening (FIG. 6A). Three had relapsing prosthetic knee infection despite suppressive antibiotics following open DAIR. Two had clinical signs of septic arthritis (FIG. 6B); the two others had fistula. No adverse events occurred during arthroscopy; all patients received daptomycin 8 mg/kg and linezolid (600 mg twice daily; 4 to 6 weeks), followed by tedizolid 200 mg/day as suppressive therapy. At 6 months, recurrence of the fistula occurred in the two patients with fistula at baseline. After 1 year follow up, the outcome was favorable in the two septic arthritis patients, with disappearance of clinical signs of septic arthritis (FIG. 6C). This favorable outcome supports that CF-301 may be efficaciously used during arthroscopic DAIR in patients with relapsing MDR Staphylococcus infections to improve the efficacy of suppressive antibiotics and to avoid considerable loss of function.

### Example 6. Efficacy of Pp296 in Rat Osteomyelitis Model

Infections of methicillin-resistant Staphylococcus aureus (IDRL-6169; isolated from a patient with a prosthetic hip infection) were established in Sprague Dawley rats according to the protocol as described in Karau et al., Exebacase in Addition to Daptomycin Is More Active than Daptomycin or Exebacase Alone in Methicillin-Resistant Staphylococcus aureus Osteomyelitis in Rats, Antimicrob. Agents Chemother. 2019 Sep. 23; 63(10). Specifically, osteomyelitis was established in the rats by bending the knee joint, inserting a 21G needle into the tibial process, and injecting 10 μl arachidonic acid and 50 μl of about 10⁶-10⁸ colony forming units (cfu) suspension of methicillin-resistant Staphylococcus aureus IDRL-6169.

The following six treatment groups were identified: (1) control/no treatment (n=18); (2) 60 mg/kg daptomycin (DAP) administered subcutaneously twice daily for 4 days (n=17); (3) 40 mg/kg pp296 administered intravenously daily for 4 days (n=17); (4) 40 mg/kg pp296 administered intravenously daily for 4 days plus 60 mg/kg DAP administered subcutaneously twice daily for 4 days (n=17); (5) 100 mg/kg pp296 administered intravenously once as a single dose on treatment day 1 (n=17); and (6) 100 mg/kg pp296 administered intravenously once as a single dose on treatment day 1 plus 60 mg/kg DAP administered subcutaneously twice daily for 4 days (n=17). When daptomycin was administered together with pp296, it was given 15 minutes prior to pp296, which was maintained on ice.

Animals were sacrificed twelve hours after the last treatment was administered, and the tibia were collected, weighed, and cryopulverized for quantitative bacterial culture. The log₁₀ CFU counts/g of tibia bone was determined using the Wilcoxon rank sum test, adjusted with a false discovery rate approach. The results are shown below in Table 4.

TABLE 4
Log₁₀ CFU/g of rat tibia bone

| | Mean | Median | | Mean log₁₀ |
|---|---|---|---|---|
| | log₁₀ | log₁₀ | | CFU/g |
| Treatment group | CFU/g | CFU/g | SD | reduction |
| (1) No treatment | 5.42 | 5.31 | 1.07 | — |
| (2) DAP 60 mg/kg | 4.29 | 4.79 | 1.89 | −1.14 |
| (3) pp296 40 mg/kg | 4.94 | 5.27 | 1.15 | −0.49 |
| (4) pp296 40 mg/kg + | 4.84 | 4.71 | 0.50 | −0.59 |
| DAP | | | | |
| (5) pp296 100 mg/kg | 4.81 | 4.99 | 1.59 | −0.61 |
| (6) pp296 100 mg/kg + | 3.67 | 3.97 | 1.50 | −1.76 |
| DAP | | | | |

The results indicate that a single dose of 100 mg/kg pp296 synergized with daptomycin to decrease the mean log₁₀ CFU by 1.76 CFU/g compared to untreated controls and 0.62 CFU/g compared to daptomycin alone. This reduction was significant compared to untreated controls (P=0.003), as well as pp296 single and daily doses alone (i.e., without daptomycin) (P=0.0210 and P=0.0175, respectively). These results for pp296 are comparable to the results obtained for CF-301. For example, a single dose of 40 mg/kg of CF-301 in combination with daptomycin resulted in log₁₀ CFU/g decrease of 0.52 compared to daptomycin alone.

Additionally, body weights of the animals were monitored as a marker of general health status during the study. The mean body weight of the animals at the time of surgery (day 1), immediately prior to treatment (day 8), and at the time of sacrifice (day 12) are shown below in Table 5.

TABLE 5
Mean body weight of rats

| | Body | Body | Body |
|---|---|---|---|
| | weight | weight | weight |
| Treatment group | on Day 1 | on Day 8 | on Day 12 |
| (2) No treatment | 337 | 308 | 314 |
| (2) DAP 60 mg/kg | 339 | 312 | 312 |
| (3) pp296 40 mg/kg | 342 | 313 | 311 |
| (4) pp296 40 mg/kg + | 335 | 307 | 298 |
| DAP | | | |
| (5) pp296 100 mg/kg | 344 | 312 | 312 |
| (6) pp296 100 mg/kg + | 342 | 309 | 308 |
| DAP | | | |

It was noted that there was a marked decrease in animal weights over the first 7 days after infection before treatment started. Little to no weight loss was noted during the four days of treatment in all treatment groups.

Pathological slides revealed hypercellular marrow with an increase in neutrophils in all groups with the exception of the 40 mg/kg daily dosing of pp296 (Treatment Group 3), which showed only possible hypercellularity. These findings are consistent with acute osteomyelitis, and no appreciable differentiation between groups could be made.

1. A method of treating or preventing a bone or joint infection, which method comprises:
administering to a subject in need thereof a therapeutically effective amount of a PlySs2 lysin comprising the amino acid sequence of SEQ ID NO: 1 or a variant thereof having at least 80% identity to SEQ ID NO: 1,
wherein the variant comprises bactericidal and/or bacteriostatic activity against the Gram-positive bacteria, and
wherein the bone or joint infection comprises a Gram-positive bacteria.

2. The method of claim 1, wherein the bone or joint infection comprises a biofilm.

3. The method of claim 1, wherein the bone or joint infection comprises osteomyelitis, a prosthetic joint infection or a native joint infection.

4. The method of claim 3, wherein the osteomyelitis is chronic osteomyelitis or the prosthetic joint infection is a recurring prosthetic joint infection.

5. The method of claim 3, wherein the bone or joint infection is osteomyelitis and wherein the osteomyelitis is acute osteomyelitis, exogenous osteomyelitis, implant-associated osteomyelitis or haematogenous osteomyelitis.

6-7. (canceled)

8. The method of claim 3, wherein the prosthetic joint infection comprises a prosthetic hip, shoulder, elbow, ankle or knee infection.

9. The method of claim 1, wherein the subject suffers from obesity, diabetes mellitus, rheumatoid arthritis or is elderly.

10. The method of claim 1, wherein the method of treatment further comprises Debridement and Implant Retention (DAIR).

11. A method for prevention or disruption of a biofilm formed in a synovial fluid of a subject comprising:
administering to a subject in need thereof a therapeutically effective amount of a PlySs2 lysin comprising the amino acid sequence of SEQ ID NO: 1 or a variant thereof having at least 80% identity to SEQ ID NO: 1,
wherein the variant comprises bactericidal and/or bacteriostatic activity against the Gram-positive bacteria,
wherein the biofilm is formed by a Gram-positive bacteria.

12. The method of claim 1, wherein the administering step further comprises co-administering a therapeutically effective amount of one or more antibiotics.

13. The method of claim 12, wherein the one or more antibiotics is/are selected from the group consisting of a beta-lactam, an aminoglycoside, a glycopeptide, an oxazolidinone, a lipopeptide and a sulfonamide.

14. The method of claim 12, wherein the one or more antibiotics comprises rifamycin, an aminoglycoside, a sulfonamide, and/or tedizolid.

15. The method of claim 12, wherein the one or more antibiotics comprises vancomycin or daptomycin.

16. (canceled)

17. The method of claim 1, wherein the Gram-positive bacteria comprise Staphylococcus bacteria, Enterococcus bacteria and/or Streptococcus bacteria.

18. The method of claim 17, wherein the Staphylococcus bacteria comprises Staphylococcus aureus.

19. (canceled)

20. The method of claim 1, wherein the Gram-positive bacteria comprise coagulase-negative staphylococci.

21. The method of claim 20, wherein the coagulase-negative staphylococci comprises at least one of Staphylococcus simulans, Staphylococcus caprae, Staphylococcus lugdunensis and/or Staphylococcus epidermidis.

22. The method of claim 1, wherein the Gram positive bacteria comprise multidrug-resistant Staphylococcus epidermidis.

23. The method of claim 1, wherein the PlySs2 lysin comprises the amino acid sequence of SEQ ID NO: 1 without the initial methionine residue.

24. The method of claim 1, wherein the PlySs2 lysin variant comprises at least one of the following amino acid sequences: SEQ ID NO: 3-17.

25. The method of claim 24, wherein the PlySs2 lysin variant comprises the amino acid sequence of SEQ ID NO: 6.

26. The method of claim 1, wherein the PlySs2 lysin has at least 90% identity to the polypeptide of SEQ ID NO: 1.

27. (canceled)

28. The method of claim 1, wherein the Gram-positive bacteria comprise Methicillin-resistant Staphylococcus aureus.

29. (canceled)

30. The method of claim 1, wherein the PlySs2 is administered during arthroscopy.

31. (canceled)

32. The method of claim 1, wherein the Gram-positive bacteria comprises multidrug-resistant Gram-positive bacteria.

33. The method of claim 11, wherein the Gram-positive bacteria comprises multidrug-resistant Gram-positive bacteria.

34. The method of claim 1, wherein the PlySs2 lysin or variant thereof is administered intravenously in a single dose.

35. The method of claim 1, wherein the PlySs2 or variant thereof is formulated as a single bolus for injection.

36. The method of claim 1, wherein the Gram-positive bacteria has entered into an osteoblast.

37. The method of claim 5, wherein the exogenous osteomyelitis is implant-associated osteomyelitis.

38. The method of claim 37, wherein the implant-associated osteomyelitis is from an implant selected from a metal plate, a pin, a rod, a wire and/or a screw.

39. A method of treating a relapsing multidrug-resistant staphylococcal prosthetic joint infection, which method comprises:
administering to a subject in need thereof a therapeutically effective amount of a PlySs2 lysin comprising the amino acid sequence of SEQ ID NO: 1 or a variant thereof having at least 80% identity to SEQ ID NO: 1,
wherein the variant comprises bactericidal and/or bacteriostatic activity against the Gram-positive bacteria.

40. The method of claim 39, wherein the administering comprises arthroscopically administering a single dose of PlySs2 and an antibiotic,
wherein the PlySs2 comprises SEQ ID NO: 1 without the initial methionine residue, and
wherein the relapsing multidrug-resistant staphylococcal prosthetic joint infection is a prosthetic knee infection.

41. A composition comprising a therapeutically effective amount of a PlySs2 lysin comprising the amino acid sequence of SEQ ID NO: 1 or a variant thereof having at least 80% identity to SEQ ID NO: 1, and one or more antibiotic(s) comprising an aminoglycoside, sulfonamide, rifamycin and/or tedizolid,
wherein the variant comprises bactericidal and/or bacteriostatic activity against Gram-positive bacteria,
wherein the PlySs2 lysin and/or the variant thereof and/or the one or more antibiotics is/are formulated at a dosage below the minimal inhibitory concentration (MIC) dose.

42. The composition of claim 41, wherein the PlySs2 lysin comprises SEQ ID NO: 1 without the initial methionine residue.

43. The method of claim 1, wherein the PlySs2 lysin or variant thereof is administered in an amount of about 0.25 mg/kg.
